(19) United States
(12) Patent Application Publication
(10) Pub. No.: US 2024/0180438 A1
(43) Pub. Date: Jun. 6, 2024

(54) MEDICAL CARE ASSISTANCE SYSTEM, MEDICAL CARE ASSISTANCE DEVICE, AND RECORDING MEDIUM

(71) Applicant: OMRON HEALTHCARE Co., Ltd., Kyoto (JP)

(72) Inventors: Fumihiko NAKAMURA, Kyoto (JP); Ai KANAZAWA, Kyoto (JP); Hiroshi USUI, Kyoto (JP)

(21) Appl. No.: 18/442,601

(22) Filed: Feb. 15, 2024

Related U.S. Application Data
(63) Continuation of application No. PCT/JP2023/007723, filed on Mar. 2, 2023.

(30) Foreign Application Priority Data
Mar. 31, 2022 (JP) 2022-060886

(51) Int. Cl. A61B 5/024; A61B 5/00

(52) U.S. Cl. CPC A61B 5/024; A61B 5/7282; A61B 5/743

(57) ABSTRACT

A medical care assistance system comprising a biological information acquisition means configured to acquire patient biological information of a patient to be managed including a heart rate and a pulse rate of one or more opportunities for each day, a daily measurement value calculation means configured to calculate one heart rate and one pulse rate of the patient in the one opportunity for each day based on the biological information and a predetermined calculation rule, a medical care assistance image generation means configured to generate a medical care assistance image including a heart rate/pulse rate graph in which the one heart rate and the one pulse rate for each day are plotted in one graph area having a time for one axis and a rate for another axis, and an output means configured to output the medical care assistance image.

### CROSS-REFERENCE TO RELATED APPLICATIONS

This application is the U.S. national stage application filed pursuant to U.S.C. 365(c) and 120 as a continuation of International Patent Application No. PCT/JP2023/007723, filed Mar. 2, 2023, which application claims priority to Japanese Patent Application No. 2022-060886, filed Mar. 31, 2022, which applications are incorporated herein by reference in their entireties.

### TECHNICAL FIELD

The present invention belongs to a technical field related to healthcare, and relates to a medical care assistance system, a medical care assistance device, and a program.

### BACKGROUND ART

In recent years, there has been proposed a system that continuously acquires and records biological information of a patient and indicates transition over time in the biological information to assist medical care by a physician (for example, Patent Document 1).

In Patent Document 1, a medical information processing system is disclosed which stores vital data of a patient in association with time, displays the vital data in time series, and calculates and displays statistical information related to the vital data displayed in time series. According to this system, an operator such as a physician can easily comprehend the tendency of the vital data of the patient, comprehend the condition of the patient, and determine the type and dosage of medication to be prescribed to the patient.

By using such a system, it is possible to reduce a burden on a physician in the medical care of a patient, in particular the patient having a chronic disease, and if an appropriate treatment strategy can be quickly determined, the effect will be exerted on the patient.

### CITATION LIST

### Patent Literature

Patent Document 1: JP 2019-24943 A

### SUMMARY OF INVENTION

### Technical Problem

There may be a case where an error occurs at the time of measurement of vital data. Specifically, it is a case where a value greatly different from a normally expected value is acquired, a case where a value cannot be acquired, or the like. Causes of such a phenomenon include a failure in attachment of a measuring device or a body motion at the time of measurement (measurement error), a deviation of a measurement value due to a disease condition of a patient (for example, a deviation between a heart rate and a pulse rate which should be the same when a healthy person correctly performs measurement at the same time), and the like. In any case, these states require attention. Even in the technique described in Patent Document 1, when such defective vital data is included or a data set with lacking is used, the reliability of statistical information to be provided is degraded.

In addition, in a case where there is a deviation of the measurement value due to the disease condition of the patient, this case may be considered that there is a symptom or a change in the disease condition to be detected. Therefore, it is useful for appropriate diagnosis to be able to comprehend such a deviation at an early stage. In a case of a measurement error in which the measurement itself is not normally performed, it is enough to simply perform measurement again. However, in a case where a device (for example, a wearable device or the like) that automatically performs series of processing from measurement to recording and transmission of measurement data and the like according to a rule is used, there is a possibility that the patient can not notice that the measurement is not normally completed.

In view of the above-described problem, the present invention relates to a system related to medical assistance, and an object of the present invention is to provide a technique for assisting accurate diagnosis by reducing the load of medical care on a physician.

### Solution to Problem

The present invention adopts the following configurations to solve the above-described problems. That is, a medical care assistance system including a biological information acquisition means configured to acquire biological information of a patient including a heart rate of one or more opportunities and a pulse rate of one or more opportunities of a patient to be managed, for each day, a daily measurement value calculation means configured to calculate one heart rate and one pulse rate of the patient for each day based on the biological information and a predetermined calculation rule, a medical care assistance image generation means configured to generate a medical care assistance image including a heart rate/pulse rate graph in which the one heart rate and the one pulse rate for each day are plotted in one graph area having a time for one axis and a rate for another axis, and an output means configured to output the medical care assistance image.

Here, the “heart rate” is the number of beats of the heart, and the “pulse rate” is the number of beats of a blood vessel of the body (of a site to be measured). The term “one opportunity” refers to a set of measurement timings for one piece of biological information, such as “morning (within one hour after getting up)” and “evening (before going to bed)” in the guidelines for diagnosis and treatment of high blood pressure. To be more specific, for example, it can be specified by setting a certain time period as “one opportunity=10 minutes (from the start of measurement of the first biological information)”. In addition, a series of biological information measured within the time is collectively referred to as “biological information obtained in one opportunity”. In addition, “one . . . in one opportunity” means that only one measurement value is calculated even when a plurality of measurements is performed on the same biological information in one opportunity and a plurality of measurement values are obtained. A specific example is that one representative measurement value is selected from a plurality of measurement values in one opportunity, or an average value of a plurality of measurement values is obtained as one measurement value.

According to such a configuration, the physician can easily check the transition of the systole function of the patient by referring to the output medical care assistance image. In addition, by plotting the heart rate and the pulse rate in the same graph area, it is possible to visually recognize and easily comprehend the deviation between the measurement values of the heart rate and the pulse rate at the same measurement opportunity, and it is possible to more appropriately perform the medical care of the patient. Further, even if there is a measurement error in either the measurement of the heart rate or the measurement of the pulse rate, the systole function of the patient can be diagnosed by the other numerical value.

The biological information may include the presence or absence of atrial fibrillation, and the medical care assistance image generation means may be configured to generate the medical care assistance image including the heart rate/pulse rate graph plotted together with a heart rate when the atrial fibrillation is detected. With such a configuration, since the measurement value of the heart rate when the atrial fibrillation is detected is separately plotted in the graph area, the physician can consider the change in the cardiac function of the patient including the occurrence state of the atrial fibrillation at the time of diagnosis.

In addition, in a case where the atrial fibrillation is present, the medical care assistance image generation means may be configured to generate the medical care assistance image in which a mark indicating the presence of the atrial fibrillation and/or a heart rate when the atrial fibrillation is detected are displayed together on the heart rate/pulse rate graph. In addition, the biological information acquisition means may be also configured to acquire the presence or absence of arrhythmia for each day of the patient, and the medical care assistance image generation means may be configured to, when the arrhythmia is present, generate the medical care assistance image in which a mark indicating the presence of the arrhythmia is displayed together with the heart rate/pulse rate graph. According to such a configuration, the physician can easily check the occurrence frequency of atrial fibrillation or arrhythmia and the transition of the occurrence frequency by referring to the medical care assistance image being output.

In addition, the daily measurement value calculation means determines, with respect to the heart rate and the pulse rate in the one opportunity, a measurement value of first measurement as a measurement value of the one opportunity when further measurement is not performed within 10 minutes from the first measurement, or determines an average value of all measurement values performed within 10 minutes as the measurement value of the one opportunity when further measurement is performed within 10 minutes from the first measurement. According to such a rule, it is possible to easily obtain one measurement value even in a case where only one measurement is performed in one opportunity or in a case where a plurality of measurements is performed, and it is possible to suppress a load on the system.

In addition, when the biological information acquisition means acquires the heart rate and the pulse rate at a plurality of measurement opportunities per one day, the daily measurement value calculation means may select one opportunity from the plurality of measurement opportunities and calculate the one heart rate and the one pulse rate for each day based on measurement values of the heart rate and the pulse rate in the selected opportunity.

In addition, when the biological information acquisition means acquires the heart rate and the pulse rate at a plurality of measurement opportunities per one day, the daily measurement value calculation means may select a measurement opportunity at a predetermined timing as the one opportunity in the one day, and calculate the one heart rate and the one pulse rate based on a measurement value measured at the one opportunity.

In addition, when the biological information acquisition means acquires the heart rate and the pulse rate at a plurality of measurement opportunities per one day, the daily measurement value calculation means may select a measurement opportunity in which a deviation between the heart rate and the pulse rate in one measurement opportunity is the largest as the one opportunity in the one day, and calculate the one heart rate and the one pulse rate based on a measurement value measured at the one opportunity.

In addition, when the biological information acquisition means acquires the heart rate and the pulse rate at a plurality of measurement opportunities per one day, the daily measurement value calculation means may select a measurement opportunity in which a time difference between measurement times of the heart rate and the pulse rate in one measurement opportunity is the smallest as the one opportunity in the one day, and calculate the one heart rate and the one pulse rate based on a measurement value measured at the one opportunity.

In addition, when the biological information acquisition means acquires the heart rate and the pulse rate at a plurality of measurement opportunities per one day and acquires a plurality of the heart rates or the pulse rates in each opportunity, the daily measurement value calculation means may select a measurement opportunity in which a difference between a plurality of the heart rates or between a plurality of the pulse rates acquired in each opportunity is the smallest as the one opportunity in the one day, and calculate the one heart rate and the one pulse rate based on a measurement value measured at the one opportunity.

In addition, the biological information acquisition means may also acquire information related to whether or not a measurement state is likely to adversely affect the measurement at the time of measurement of the heart rate and the pulse rate. In addition, when the heart rate and the pulse rate displayed in the heart rate/pulse rate graph are calculated using a measurement value measured in a measurement state that is likely to adversely affect the value, the medical care assistance image generation means may generate the medical care assistance image including the heart rate/pulse rate graph allowing to identify that the heart rate and pulse rate are calculated using a measurement value measured in a measurement state that is likely to adversely affect the value. Specifically, for example, in a case where a body motion at the time of measurement or an attachment defect of the device is detected, a mark indicating the detection may be displayed together, or a color, a shape, or the like of a point at which a measurement value is plotted may be changed. According to this, the physician can easily identify whether or not the displayed heart rate and pulse rate are reliable values.

The biological information acquisition means may also acquire information related to whether or not a measurement state is likely to adversely affect the measurement at the time of measurement of the heart rate and the pulse rate, when the biological information acquisition means acquires the heart rate and the pulse rate at a plurality of measurement opportunities per one day, the daily measurement value calculation means may use, as the one opportunity in the one day, a measurement opportunity in which there is no information indicating that the measurement state is likely to adversely affect the measurement at the time of measurement of the heart rate and the pulse rate, and calculate the one heart rate and the one pulse rate based on a measurement value measured in the one opportunity.

The present invention can also be considered as a medical care assistance device that includes the biological information acquisition means, the daily measurement value calculation means, and the medical care assistance image generation means, and the medical care assistance device constitutes at least a part of the medical care assistance system.

Further, the present invention can also be considered as a program for causing a computer to function as such a medical care assistance device, and a computer-readable recording medium recording such a program in a non-transitory manner. Also, the configurations and processing described above can be combined with one another to constitute the present invention unless the combination leads to contradiction.

### Advantageous Effects of Invention

According to the present invention, it is possible to provide a technique for a system related to medical assistance, which is capable of reducing a workload on a medical professional to diagnose severity of a disease of medical care target for a patient.

### BRIEF DESCRIPTION OF DRAWINGS

FIG. 1 is a schematic diagram illustrating a configuration of a medical care assistance system according to an example.

FIG. 2 is a block diagram illustrating a functional configuration of a server device according to the example.

FIG. 3 is a diagram illustrating an example of a data table according to the example.

FIG. 4 is a block diagram illustrating a functional configuration of a physician-side terminal according to the example.

FIG. 5 is a first diagram illustrating an example of a medical care assistance image output on a physician-side terminal.

FIG. 6A is a second diagram illustrating an example of the medical care assistance image output on the physician-side terminal. FIG. 6B is a third diagram illustrating an example of the medical care assistance image output on the physician-side terminal.

FIG. 7A is a fourth diagram illustrating an example of the medical care assistance image output on the physician-side terminal. FIG. 7B is a fifth diagram illustrating an example of the medical care assistance image output on the physician-side terminal.

FIG. 8A is a sixth diagram illustrating an example of the medical care assistance image output on the physician-side terminal. FIG. 8B is a seventh diagram illustrating an example of the medical care assistance image output on the physician-side terminal.

FIG. 9 is an eighth diagram illustrating an example of the medical care assistance image output on the physician-side terminal.

FIG. 10 is a block diagram illustrating a functional configuration of a patient-side terminal according to the example.

FIG. 11A is a first diagram illustrating an example of a user interface displayed on the patient-side terminal. FIG. 11B is a second diagram illustrating an example of the user interface displayed on the patient-side terminal.

FIG. 12 is a diagram illustrating a flow of information exchange and processing performed in the medical care assistance system according to the example.

FIG. 13 is a diagram illustrating an outline of a medical care assistance system according to another aspect.

### DESCRIPTION OF EMBODIMENTS

### Example 1

Embodiments of the present invention will be specifically described below with reference to the drawings. However, it should be noted that the dimension, shape, relative arrangement and the like of the components described in this embodiment are not intended to limit the scope of this invention to them alone, unless otherwise stated.

### System Configuration

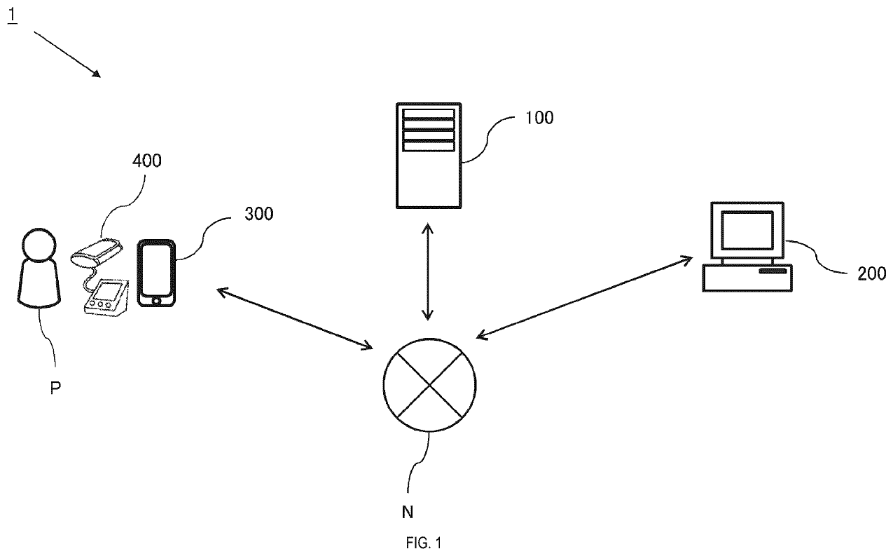

FIG. 1 is a schematic diagram illustrating a configuration of a medical care assistance system 1 according to the present example. As illustrated in FIG. 1, the medical care assistance system 1 is configured by including a server device 100, a physician-side terminal 200 used by a physician, and a patient-side terminal 300 and a measuring device 400 that are used by a patient P. All of these in the system configuration can communicate with one another via a communication network N.

The medical care assistance system 1 according to the present example is a system related to medical care, which assists a physician to perform treatment on a patient by transmitting measurement values of biological information such as a heart rate, a pulse rate, a blood pressure value, and a weight measured by the patient at home to the server device 100 via the communication network N, processing the information, and providing the information to a medical professional.

A patient who is determined that he or she needs continuous monitoring of biological information by receiving, for example, a definitive diagnosis of the heart failure or the like, starts treatment in accordance with a diagnosis by the physician, continuously measures biological information at home by himself or by herself, and records subjective symptoms in daily life. The medical care assistance system 1 collects information related to the measured value and the subjective symptom, generates a medical care assistance image for a medical professional such as a physician to refer to regarding medical care of the patient based on the information being collected, and outputs the medical care assistance image via an output means. The medical care assistance image is referred to at the time of medical examination of a patient and is appropriately referred to as necessary for medical care.

The medical care assistance system 1 may indicate alert information of the medical care assistance image when the collected measurement value of the patient satisfies an alert condition set in advance. Further, the alert signal may be transmitted to an information processing terminal, a portable communication terminal, or the like possessed by the physician. Hereinafter, the respective configurations of the system will be described in detail.

### Server Device

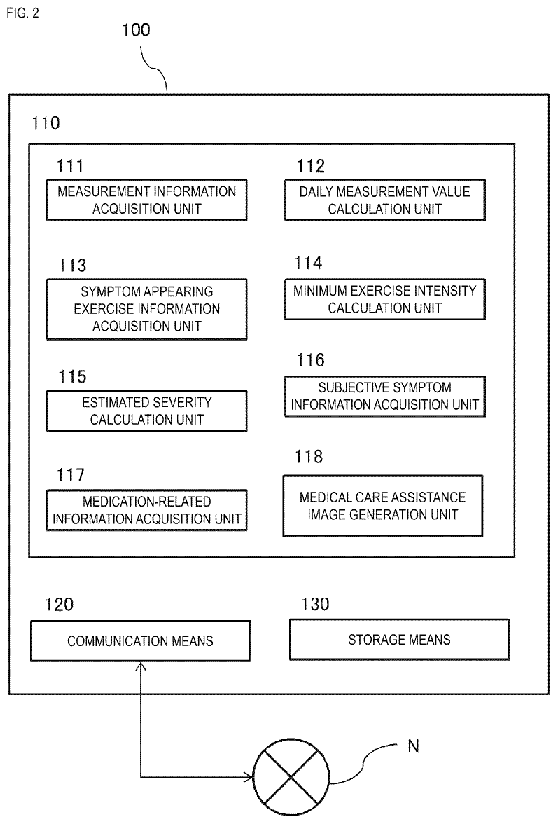

FIG. 2 is a block diagram illustrating a functional configuration of the server device 100. The server device 100 is constituted of a general server computer and, as illustrated in FIG. 2, includes a control unit 110, a communication means 120, and a storage means 130.

The control unit 110 is a means that manages the control of the server device 100, and is constituted of a processor, such as a central processing unit (CPU) or a digital signal processor (DSP). The control unit 110 includes, as functional modules related to biological information management, functional units such as a measurement information acquisition unit 111, a daily measurement value calculation unit 112, a symptom appearing exercise information acquisition unit 113, a minimum exercise intensity calculation unit 114, an estimated severity calculation unit 115, a subjective symptom information acquisition unit 116, a medication-related information acquisition unit 117, and a medical care assistance image generation unit 118. These respective functional units will be described in detail below.

The communication means 120 is a communication means configured to connect the server device 100 to the communication network N, and is constituted including, for example, a communication interface board and a wireless communication circuit for wireless communication.

Although not illustrated, the storage means 130 includes a main storage unit, such as a read only memory (ROM) or a random access memory (RAM), and an auxiliary storage unit, such as an EPROM, a hard disk drive (HDD), a solid state device (SSD), or a removable medium. The auxiliary storage unit stores an operating system (OS), various programs, and the like. By the stored programs being loaded into a work area of the main storage unit and executed, and the respective component units and the like being controlled by the execution of the programs, the respective functional units that meet predetermined purposes can be realized.

As will be described below, the measurement information acquisition unit 111 acquires, via the communication network N, measurement values of biological information such as a heart rate, a pulse rate, and a blood pressure value measured by the patient P with the measuring device 400, and stores the measurement values in the storage means 130. These measurement values can be acquired by various known measuring devices. Further, as the measuring device, separate devices corresponding to each biological information may be used, or a measuring device that can acquire different measurement values by one device (one measurement), for example, an upper arm-type oscillometric blood pressure monitor that can acquire a blood pressure value and a pulse rate may be used.

In addition, in a case where a specific symptom such as atrial fibrillation (AF) or a suspicion thereof is detected at the time of heart rate measurement, the measurement information acquisition unit 111 acquires information of the specific symptom or the suspicion together with the heart rate and stores the information in the storage means 130. In addition, when a specific symptom such as arrhythmia or a suspicion thereof is measured at the time of pulse rate measurement, the measurement information acquisition unit 111 also acquires information indicating measurement of the specific symptom or the suspicion and stores the information in the storage means 130. The information of the measurement value acquired by the measurement information acquisition unit 111 includes time information at which the measurement is performed and information about a place at which the measurement is performed (for example, home or medical examination room). In addition, information (for example, body motion detected at the time of measurement, information indicating a defect in an attachment state of a measurement state, or the like) detected by the measuring device 400 is also acquired, which is related to whether or not a measurement state is likely to adversely affect the measurement at the time of measurement.

The daily measurement value calculation unit 112 calculates the value of the biological information of the patient P for each day based on the measurement value stored in the storage means 130. It should be noted that the calculation method of the value calculated here varies depending on the type of the target biological information or disease. For example, even when measurement is performed a plurality of times per day, a biological information value per day may be determined as one value (a heart rate, a pulse rate, or the like to be described below), or a plurality of measurement values in different measurement time zones may be determined as measurement values in one day (so as to be identifiable as such). Hereinafter, a case of calculating daily measurement values of a heart rate and a pulse rate in a case where a patient with heart failure is a target will be described.

The daily measurement value calculation unit 112 calculates one heart rate and one pulse rate in one opportunity of the patient P for each day based on the measurement values stored in the storage means 130, and stores the calculated heart rate and pulse rate in the storage means 130. The term “one opportunity” refers to a set of measurement timings for one piece of biological information, such as “morning (within one hour after getting up)” and “evening (before going to bed)” in the guidelines for diagnosis and treatment of high blood pressure. In the present example, with respect to such a measurement timing, for example, a certain constant time span is set as “one opportunity=10 minutes (from the start of the measurement of the first biological information)”, and a series of a plurality of pieces of biological information (including different kinds of biological information) measured within the time span are collectively set as “biological information obtained in one opportunity”. That is, regarding the measurement of the biological information, when the measurement is performed a plurality of times within the certain constant period of time, the plurality of measurements is collectively set as the measurement of one opportunity, and the pieces of biological information measured a plurality of times correspond to the biological information obtained in one opportunity. Here, in any of a case where only the identical biological information is measured a plurality of times, a case where different pieces of biological information are each measured once, and a case where different pieces of biological information are each measured once or more, the plurality of measurements corresponds to the measurement in one opportunity if all the measurements are performed within a certain constant period of time.

On the other hand, even when one piece of biological information is measured twice, if the two measurements are not performed within a certain constant period of time (for example, once at the time of getting up in the morning and once at the time before going to bed at night), the two pieces of biological information correspond to the biological information measured at different opportunities (related to the measurement of two opportunities).

Here, the calculation of the daily measurement value by the daily measurement value calculation unit 112 will be specifically described using the heart rate as an example. First, in a case where the heart rate is measured only once a day and only the measurement value is stored in the storage means 130, the daily measurement value calculation unit 112 sets the measurement value as the daily heart rate. On the other hand, when a plurality of measurements is performed in one day and all of the plurality of measurements are within a predetermined time, the daily measurement value calculation unit 112 determines that all of the plurality of measurement values are measurement values within one opportunity, obtains one value (for example, an average value of the plurality of measurement values) as one heart rate in one opportunity based on the plurality of measurement values, and calculates the value as a daily heart rate. In addition, in a case where a plurality of measurements is performed in one day and the plurality of measurements is not within a predetermined time (that is, in a case where measurements for a plurality of opportunities are performed), the daily measurement value calculation unit 112 calculates a daily heart rate using a measurement value of any one opportunity among the plurality of measurement opportunities (for example, a measurement value of a measurement opportunity at a timing set in advance such as a measurement value of one opportunity at the time of getting up in the morning). In this case, the method of obtaining the measurement value of one opportunity when a plurality of measurements is performed within one opportunity is as described above. Here, the heart rate is described as an example, but the daily measurement value calculation unit 112 performs the similar calculation processing for other biological information such as a pulse rate.

The symptom appearing exercise information acquisition unit 113 acquires symptom appearing exercise information which is information including exercise contents in which a symptom related to a disease of medical care target for the patient P (here, heart failure) appears, and stores the symptom appearing exercise information in the storage means 130. Specifically, by causing the patient P to input or select exercise (physical activity) in which the patient P is aware of the symptom every predetermined period (for example, one week) by an application executed in the patient-side terminal 300 to be described below, the symptom appearing exercise information is acquired via the patient-side terminal 300.

The minimum exercise intensity calculation unit 114 calculates the minimum exercise intensity, which is the exercise intensity of the exercise having the smallest exercise intensity among the exercises in which the symptom related to heart failure has appeared within a predetermined period, based on the symptom appearing exercise information stored in the storage means 130. In the present example, the exercise intensity is indicated by the METs, and hereinafter, the exercise intensity of the exercise having the smallest exercise intensity among the exercises in which the symptom related to heart failure has appeared within a predetermined period is also referred to as a symptom appearance minimum METs. The METs is an indicator of an activity intensity indicating how many times more energy is consumed by various activities with respect to a resting state (a state of sitting quietly) as a 1 METs. Specifically, the minimum exercise intensity calculation unit 114 may hold, in the storage means 130, an exercise intensity table in which the content of exercise and the exercise intensity of the exercise are associated with each other, and may obtain the symptom appearance minimum METs by referring to the exercise intensity table. The symptom appearance minimum METs calculated here is stored in the storage means 130.

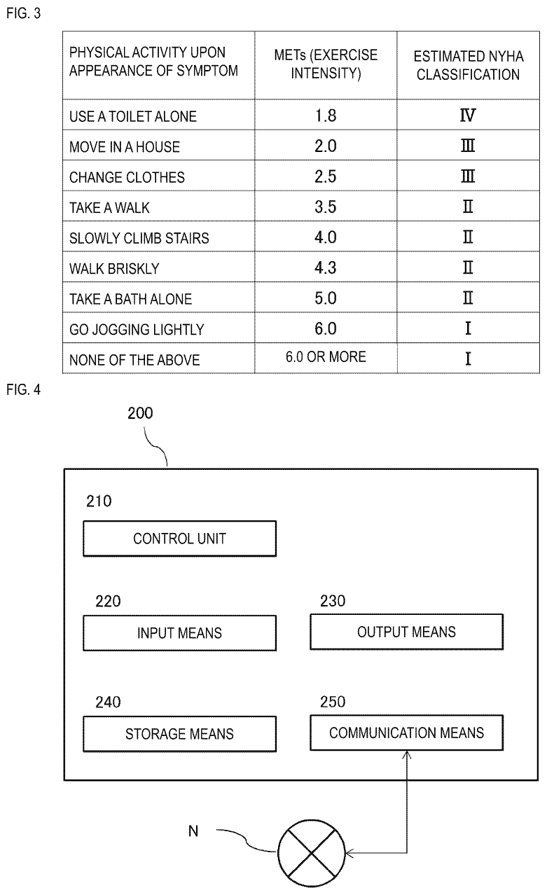

The estimated severity calculation unit 115 obtains estimated severity information indicating the severity of heart failure based on the symptom appearance minimum METs stored in the storage means 130. In the present example, the severity is based on the NYHA classification, and hereinafter, the estimated severity is also referred to as an estimated NYHA classification. For example, the estimated severity calculation unit 115 may obtain the estimated severity by referring to a data table stored in the storage means 130 in which the symptom appearance minimum METs number and the estimated NYHA classification are associated with each other. In FIG. 3, an example of a data table is illustrated in which the exercise contents, the METs (numerical values) of the exercise, and the estimated NYHA classification in the case where the METs of the exercise is the symptom appearance minimum METs number are associated with each other. Note that the exercise contents (and the corresponding METs and estimated NYHA classifications) illustrated in FIG. 3 are representative excerpts, and more exercise contents are actually defined. The estimated severity calculated here is stored in the storage means 130.

The subjective symptom information acquisition unit 116 acquires information of the presence or absence (and the type) of a symptom related to heart failure for each predetermined period (for example, every day) in the patient P, and stores the information in the storage means 130. Specifically, the information is acquired via the patient-side terminal 300 by causing the patient P to select a symptom that the patient P is aware of at a fixed time every day by an application executed in the patient-side terminal 300, similarly to the symptom appearing exercise information. Specifically, for example, a list of symptoms may be presented to allow the patient to select a symptom from the list, or input of text information related to a subjective symptom may be received as a memo or the like.

In addition, the medication-related information acquisition unit 117 acquires information regarding the presence or absence of medication and the medication rate (frequency of medication) of the patient, and stores the information in the storage means 130. In addition, information regarding the contents, frequency, and the like of side effects at the time of medication may be acquired. These pieces of information may be acquired via the patient-side terminal 300, for example, by causing the patient P to select the presence or absence of the medication of the day at a fixed time every day by the application executed in the patient-side terminal 300, similarly to the symptom appearing exercise information. Further, the medication-related information acquisition unit 117 may cooperate with an external system (for example, an electronic medical chart system) or the like, which is not illustrated, to acquire information (prescription information) of medication prescribed to the patient P.

The medical care assistance image generation unit 118 generates a medical care assistance image to be referred by a medical professional based on data output from each functional unit of the measurement information acquisition unit 111, the daily measurement value calculation unit 112, the symptom appearing exercise information acquisition unit 113, the minimum exercise intensity calculation unit 114, an estimated severity calculation unit 115, the subjective symptom information acquisition unit 116, and the medication-related information acquisition unit 117, and stored in the storage means 130. The generated medical care assistance image is transmitted to the physician-side terminal 200 via the communication network N. The medical care assistance image will be described in detail later.

### Physician-Side Terminal

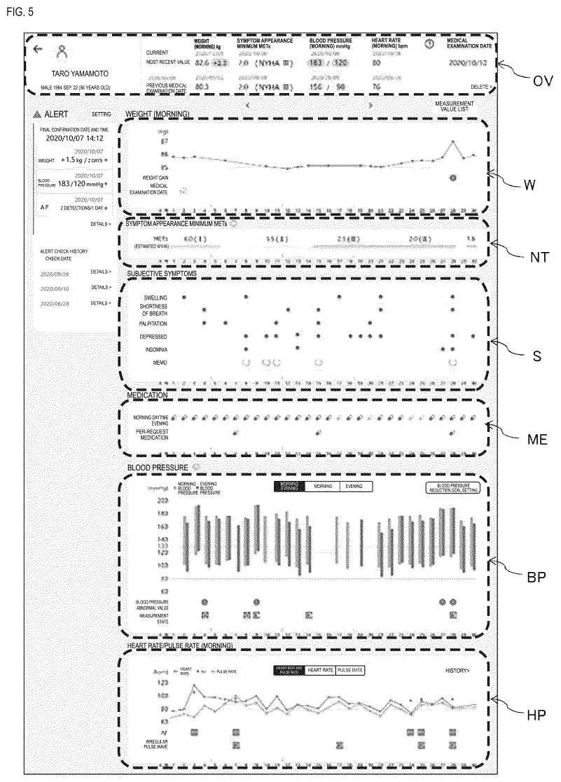

FIG. 4 is a block diagram illustrating a functional configuration of the physician-side terminal 200. The physician-side terminal 200 is a general computer, such as a fixed installation type personal computer, a portable notebook-type personal computer, or a tablet type terminal, and includes a control unit 210, an input means 220, an output means 230, a storage means 240, and a communication means 250.

The control unit 210 is a means that controls the physician-side terminal 200, and is constituted of, for example, a CPU. The input means 220 is a means that receives information input from the outside, including such as for example, a keyboard, a mouse, a touch panel, a camera, or a microphone. The output means 230 includes a liquid crystal display, a speaker, and the like. The storage means 240 is constituted similarly to the server device by including a main storage unit, an auxiliary storage unit, and the like, and stores various data, for example, an operating system (OS), various programs, and the like acquired via the communication network N. The communication means 250 is constituted including, for example, a communication interface board and a wireless communication circuit for wireless communication.

Note that, although not illustrated, the physician-side terminal may be accessible to an electronic medical chart management system. In such a case, electronic medical chart data of a patient stored in the electronic medical chart management system may be read out and the electronic medical chart data may be transmitted to the server device 100, or information transmitted from the server device 100 may be associated with the electronic medical chart data. In such a case, the physician can check the medical care assistance image via the electronic medical chart management system.

In the physician-side terminal 200, the medical care assistance image is acquired from the server device 100 via the communication network N, and such information is output to the output means 230. An example of screen (medical care assistance image) displayed on the output means 230 of the physician-side terminal 200 is illustrated in FIG. 5 to FIG. 9. FIG. 5 is an explanatory diagram illustrating an example of a medical care assistance image for one of the patients P managed by the physician who is the administrator of the terminal. As illustrated in FIG. 5, the medical care assistance image according to the present example is configured to include a plurality of regions indicating different information. Specifically, a summary information region OV, a weight information region W, an estimated NYHA classification transition region NT, a subjective symptom information region S, a medication information region ME, a blood pressure information region BP, and a heart rate/pulse rate information region HP are included. Note that the entire medical care assistance image need not be displayed on the output means, and a display region can be selected (scrolled, reduced, or enlarged) as appropriate. In addition, the medical care assistance image may be generated in a combination and order of items designated by the physician in advance.

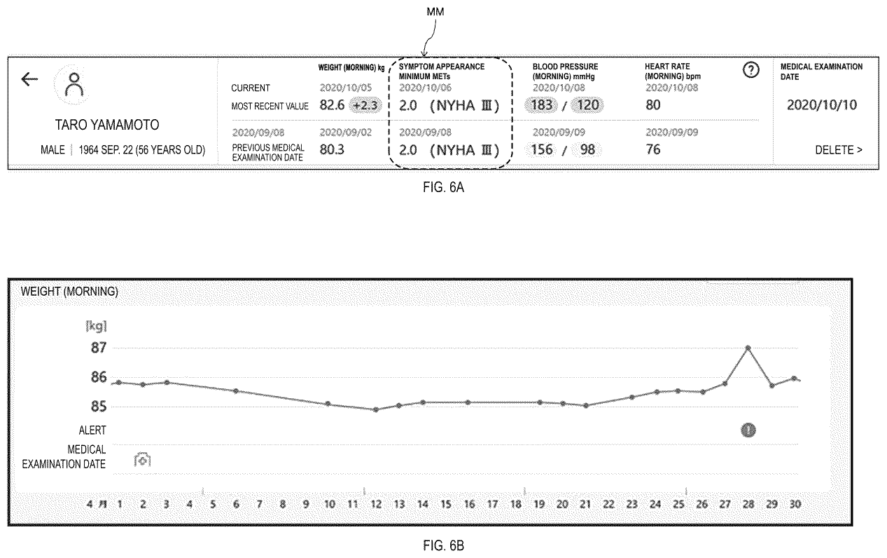

Hereinafter, information displayed in each region of the medical care assistance image will be specifically described. FIG. 6A is an enlarged view of the summary information region OV. As illustrated in FIG. 6A, the summary information region OV displays information regarding patient attributes such as patient name, gender, and age, patient information acquired most recently, and patient information at the time of the previous medical examination. In addition, Minimum METs information MM indicating the symptom appearance minimum METs (and the estimated NYHA classification) is illustrated as one piece of patient information. By such display, the physician can check the most recent symptom appearance minimum METs (and the estimated NYHA classification) of the patient P and refer to the previous symptom appearance minimum METs (and the estimated NYHA classification), and can efficiently perform a medical interview for diagnosing the severity of the patient P at the time of medical examination.

FIG. 6B is an enlarged view of the weight information region W. As illustrated in FIG. 6B, in the weight information region W, the transition of the weight of the patient P during the display period (for example, from the first day to the last day of the previous month, the past one month, the past one week, or the like) is illustrated by a graph. When the function of the heart deteriorates, water tends to accumulate in the body due to poor blood flow. Therefore, an increase in body weight (for example, an amount of increase per week) (derived from the accumulated water) is an important index related to the severity of heart failure. For this reason, alert information may be displayed in the weight information region W in a case where an increase or decrease value of the weight within a predetermined period deviates from a threshold value.

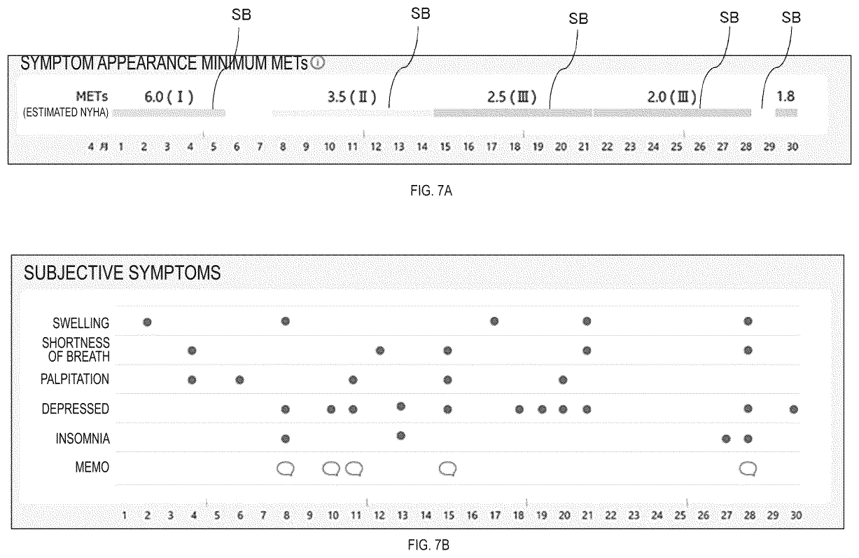

FIG. 7A is an enlarged view of the estimated NYHA classification transition region NT. As illustrated in FIG. 7A, in the estimated NYHA classification transition region NT, an estimated severity time series graph is illustrated in which the estimated NYHA classification for each predetermined period within the display period is indicated by an estimated severity display bar SB in which the difference in class can be identified by the difference in color. In addition, in the vicinity of the estimated severity display bar SB, the corresponding symptom appearance minimum METs (and the estimated NYHA classification) are displayed in text. By such a display, the physician can easily check the transition of the estimated NYHA classification of the patient within the display period, and can efficiently perform the medical interview for diagnosing the severity of the patient P at the time of medical examination.

The estimated severity display bar SB is basically displayed for each predetermined period. However, the estimated severity display bar SB may be displayed in a period shorter than the predetermined period, for example, when the acquisition timing of the information by the symptom appearing exercise information acquisition unit 113 is changed or when the predetermined period includes a schedule before the first day or after the last day of the display region.

FIG. 7B is an enlarged view of the subjective symptom information region S. As illustrated in FIG. 7B, in the subjective symptom information region S, information is displayed indicating whether or not there is a subjective symptom related to heart failure by displaying dots for each type of symptom for each day along a time series (a symptom displayed by dots is a symptom that is perceived on that day). In addition, in a case where the patient writes a daily memo via the patient-side terminal 300 described below, a display indicating this may also be displayed. By referring to such a display, the physician can easily check the transition of what kind of subjective symptom is felt by the patient P every day (the kind and the frequency).

In addition, by arranging the subjective symptom information region S and the estimated severity display bar SB side by side aligning on the same time axis, the physician can easily check the correspondence relationship between the daily transition of the subjective symptom and the transition of the estimated severity, and can efficiently comprehend the progress of the disease condition of the patient.

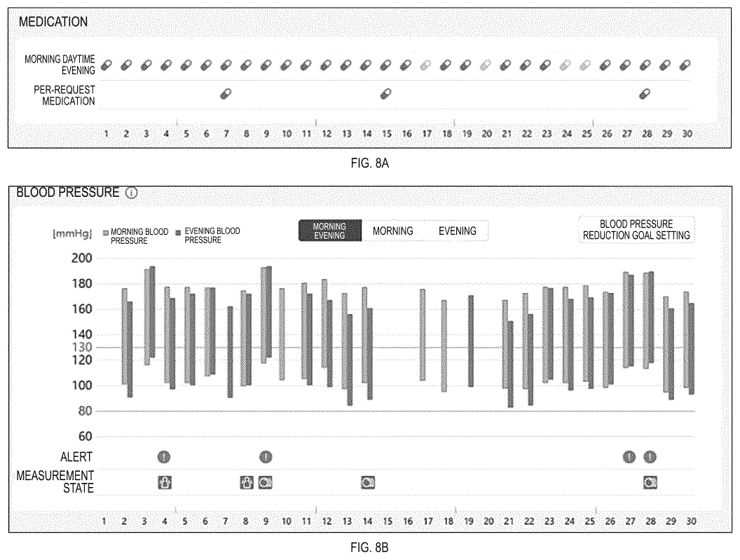

FIG. 8A is an enlarged view of the medication information region ME. As illustrated in FIG. 8A, in the medication information region ME, information of the medication of the patient within the display period (whether or not the patient has correctly taken the prescribed medication) is indicated on a daily basis by activation/deactivation of display of the capsule mark. In addition, in a case where the patient takes the per-request medication, the fact is separately displayed in the column of the date of taking the medication. In addition, when the medication is to be taken a plurality of times per day (for example, in the morning, daytime, and in the evening), a column of the presence or absence of taking the medication corresponding to each time may be provided.

Alternatively, a display related to the dosing adherence rate per day (for example, a display of marks corresponding to the number of taken medications, a display of fractions, or the like) or a display using a pie chart may be performed.

FIG. 8B is an enlarged view of the blood pressure information region BP. As illustrated in FIG. 8B, in the blood pressure information region BP, the blood pressure value in the display period is displayed on daily basis. Specifically, the blood pressure value of one opportunity is illustrated by a bar graph in which the systolic blood pressure is an upper end and the diastolic blood pressure is a lower end. When there are measurement values of two or more opportunities (for example, at the time of getting up in the morning and at the time of going to bed at night), these measurement values can be displayed in parallel as illustrated in FIG. 8B. In addition, the measurement opportunity (for example, morning/evening/others) can be displayed being identifiable by color-coded display or the like.

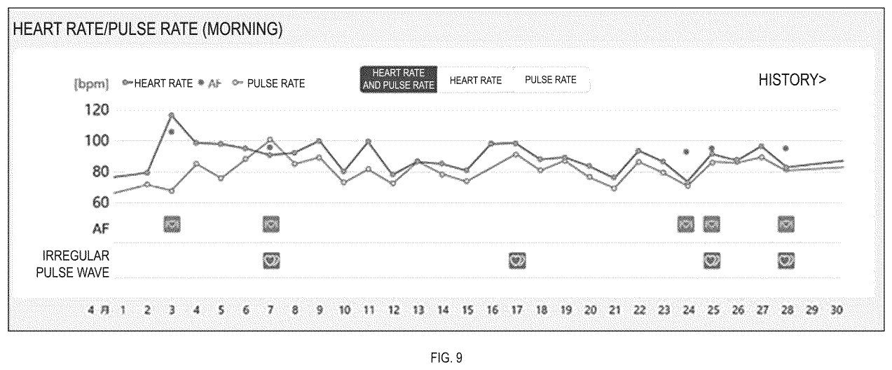

FIG. 9 is an enlarged view of the heart rate/pulse rate information region HP. As illustrated in FIG. 9, in the heart rate/pulse rate information region HP, a graph is displayed in which the daily heart rate and the daily pulse rate of each day calculated by the daily measurement value calculation unit 112, and the heart rate at the time when atrial fibrillation (AF) is detected in a case where the AF is detected are plotted on the same graph area (the X axis is the time axis, and the Y axis is the number of beats). In the graph, the daily heart rate and the daily pulse rate are one numerical value per day. However, as for the heart rate at the time when the AF is detected, in a case where the AF is detected a plurality of times per day, all of the heart rates when the AF is detected are plotted. In this manner, it is possible to distinguish between an item for which a change is desired to be tracked in time series and an item for which single information is desired to be comprehended, and to comprehend them in association with each other. Further, on a day when the AF is detected and on a day when an irregular pulse wave (arrhythmia) is detected, marks indicating the detection may be separately displayed.

In addition, in a case where the heart rate and the pulse rate displayed in the heart rate/pulse rate information region HP are calculated by using a measurement value which is measured in a measurement state that is likely to adversely affect the value, the fact may be identified. Specifically, for example, in a case where a body motion at the time of measurement or an attachment defect of the device is detected, a mark indicating the detection may be displayed together, or a color, a shape, or the like of a point at which a measurement value is plotted may be changed.

In the example illustrated in FIG. 9, there is a measurement opportunity (at the time of getting up) in which the daily measurement values of the heart rate and the pulse rate can be determined on all days of the display period, and the heart rate and the pulse rate of the measurement opportunity are plotted. On the other hand, when there is a day without a measurement opportunity in which both the heart rate and the pulse rate are appropriately measured, both the heart rate and the pulse rate may not be plotted for the day. Alternatively, a biological information value to be prioritized among the heart rate and the pulse rate may be determined in advance, and only the prioritized biological information value may be plotted (displayed). Alternatively, only the biological information value that is appropriately measured may be displayed in a mode in which the value can be identified as a reference value.

By referring to such a display, the physician can easily check the transition of the systole function of the patient P. In addition, by plotting, in the same graph area, the one heart rate and the one pulse rate calculated by the daily measurement value calculation unit 112, it is possible to visually recognize and easily comprehend the deviation between the measurement values of the heart rate and the pulse rate in the same measurement opportunity (that is, it is possible to quickly notice the possibility of the deterioration of the symptom or the disease condition to be detected), and it is possible to more appropriately perform the medical care of the patient. Further, even if there is an error in either the measurement of the heart rate or the measurement of the pulse rate, the systole function of the patient P can be diagnosed by the other numerical value. In a case where there is a difference between the heart rate and the pulse rate, it is possible to examine and determine, by considering other information, whether the difference is derived from a measurement error such as an attachment error of the device or an event to be noted such as a change in the symptom of the patient occurs. For example, in a case where a pulse rate is measured by detecting a pressure pulse wave by a cuff of an upper arm blood pressure monitor, when there is an error in the pulse rate, if a blood pressure is appropriately measured, it is possible to exclude a possibility that the error is caused by an attachment error of the device, and it is possible to estimate that the error in the pulse rate is caused by a disease condition of a patient such as arrhythmia.

The physician can efficiently acquire the information related to the patient P by referring to the medical care assistance image in which each piece of information as described above is displayed, and can reduce the burden on the patient P at the time of medical examination by eliminating futile questions from the contents of the medical interview at the time of medical examination.

### Patient-Side Terminal

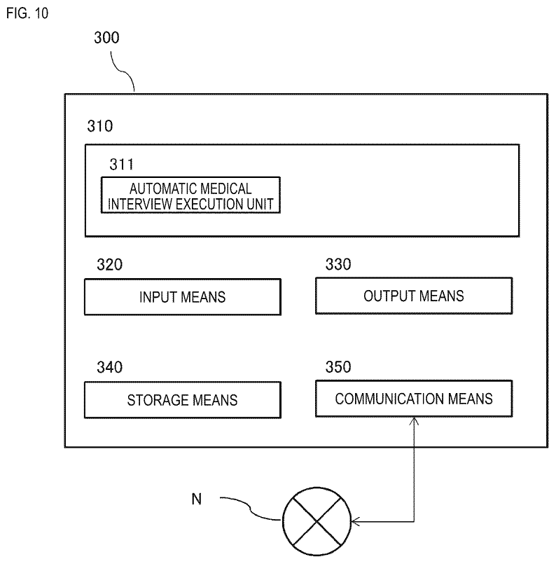

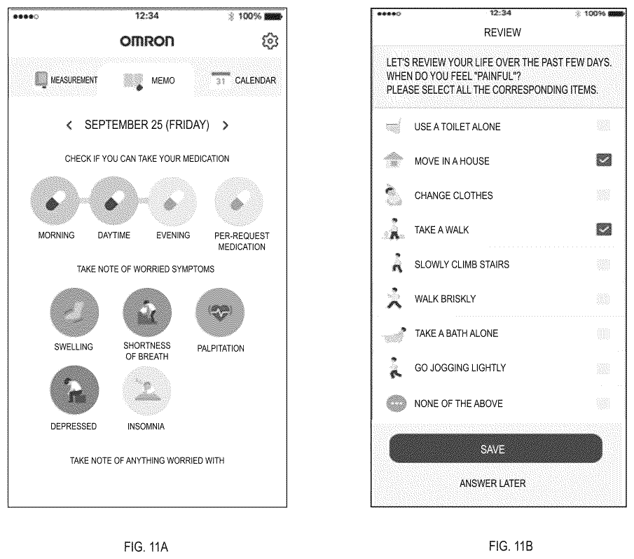

FIG. 10 is a block diagram illustrating a functional configuration of the patient-side terminal 300. The patient-side terminal 300 is, for example, a portable information processing terminal, such as a smartphone, a tablet terminal, or a wristwatch type wearable terminal, and includes a control unit 310, an input means 320, an output means 330, a storage means 340, and a communication means 350.

The control unit 310 is a means for controlling the patient-side terminal 300, and is constituted of, for example, a CPU and the like. As the input means 320, a touch panel display integrated with the output means 330 or the like can be adopted. The storage means 340 is constituted similarly to other terminals by including a main storage unit, an auxiliary storage unit, and the like, and stores various data, for example, an operating system (OS), various programs, and the like acquired via the communication network N. The communication means 250 is constituted including, for example, a wireless communication circuit for wireless communication.

The control unit 310 includes an automatic medical interview execution unit 311 as a functional module related to patient information management including symptom appearing exercise information and the like. The automatic medical interview execution unit 311 is implemented, for example, as a function provided by an application program, and receives an input of patient information via a user interface (hereinafter, referred to as UI) for requesting a user to input information to perform a medical interview. The automatic medical interview execution unit 311 may display, for example, a UI that displays a plurality of icons related to predetermined items and prompts the user to make a selection, or may adopt a format such as a so-called chatbot. Further, the application program may be stored in the storage means 340 of the patient-side terminal 300, or may be provided in the form of software as a service (Saas) in the server device 100.

The automatic medical interview execution unit 311 executes an automatic medical interview for each predetermined period set according to the information (for example, medication information, information of the presence or absence of a subjective symptom, symptom exercise information, and the like) that is requested to the patient to input. In addition, notification (screen display, voice output, or the like) for prompting the patient to input information is performed at a timing at which the automatic medical interview is executed (that is, every predetermined period).

FIG. 11A and FIG. 11B are diagrams illustrating an example of a state in which a UI provided by the automatic medical interview execution unit 311 is displayed on a screen of a smartphone as an example of the patient-side terminal 300. In FIG. 11A, a UI is illustrated which receives an input of medication information and information of the presence or absence of a subjective symptom (subjective symptom information) for each day. As illustrated in FIG. 11A, the UI is configured such that the medication information is input by selecting a medication icon for each time zone of morning, daytime, and evening, and display of the selected icon is activated. In addition, as for the subjective symptom information, the UI is also configured such that the icons indicating the respective symptoms are displayed, and an input is performed by selecting the icon of the subjective symptom. Again, the selected icon is activated for display. The automatic medical interview execution unit 311 executes an automatic medical interview processing of requesting the patient to input the medication information and the subjective symptom information at a scheduled time (for example, 21:00) every day through the screen of FIG. 11A. Note that the screen illustrated in FIG. 11A is an example of the UI related to an input of the medication information and the subjective symptom, and the user may be requested to input the subjective symptom by another UI. Specifically, for example, a list of symptoms may be presented to allow the patient to select a symptom from the list, or input of text information related to a subjective symptom may be received as a memo or the like.

In FIG. 11B, an example of a UI is illustrated which receives an input of symptom appearing exercise information for each predetermined period. As illustrated in FIG. 11B, the UI is configured such that a list of items indicating a plurality of physical activities with different exercise intensities are displayed, and an input is performed by selecting the physical activity in which a subjective symptom appears. A check mark is displayed for the selected physical activity, and thus the selected item is clearly illustrated.

The automatic medical interview execution unit 311 executes an automatic medical interview processing of requesting the patient to input symptom appearing exercise information at a timing set in advance (for example, 21:00 every Saturday) through the screen of FIG. 11B. The timing at which the automatic medical interview execution unit 311 performs the automatic medical interview processing (the timing of the notification for prompting the patient to input information) is not limited to “each specific day of the week (and time)”, that is, the timing obtained by applying a predetermined period to the calendar as described above, and may be a timing relatively calculated using the previous answer date and a predetermined period, such as “7 days after the previous automatic medical interview processing execution date (answer date)”.

In addition, when the patient does not input the information even though the automatic medical interview execution unit 311 performs the notification for prompting the input of the information, the automatic medical interview execution unit 311 may perform the notification (reminder) for prompting the input of the information again at a predetermined timing without waiting for the next predetermined period to arrive. Here, the predetermined timing may be scheduled in advance, for example, at the same time of the next day. In addition, the patient may be reminded when the patient uses the patient-side terminal 300 next time. To be specific, for example, at the time of biological information measurement by the measuring device 400 to be described below, a reminder message may be displayed together with the measurement result.

The input of information performed by the patient P via the application as described above is transmitted from the communication means 350 to the server device 100 via the communication network N. As will be described below, measurement data acquired from the measuring device 400, necessary information input by the patient P, and the like are also transmitted to the server device 100 in the same manner.

### Measuring Device

The measuring device 400 is used by the patient P for daily measurement of biological information. Here, the term “measuring device 400” is used as a concept including not only one device but also a plurality of measuring devices such as a blood pressure monitor, an electrocardiogramd a weight scale (body composition meter). Note that, the measuring device 400 includes at least a device that can measure a heart rate and a device that can measure a pulse rate. In addition, these measuring devices can detect atrial fibrillation and irregular pulse wave and acquire a measurement value and a measurement time at the time of the detection. In addition, each measuring device also detects information related to whether or not a measurement state is likely to adversely affect the measurement at the time of measurement of the biological information.

It should be noted that these measuring devices may have any form. For example, a device of a type in which an electrocardiogramd a blood pressure monitor are integrated may be used, or a body composition meter capable of performing electrocardiographic measurement may be used. In addition, the measuring device may be a stationary device or a portable device. In addition, a wearable type device which is always attached to the patient may be included.

Note that, in a case where processing from measurement to recording and transmission of measurement data and the like is automatically performed according to a rule by using the above-described wearable type device or the like, even if the measurement is not normally performed at the time of measurement, the patient may not notice that the measurement is not normally performed. Therefore, the measuring device 400 may be configured to perform a notification (image display, voice output, or the like) for prompting the patient to perform measurement again in a case where there is a measurement error (in a case where the measurement value cannot be acquired, in a case where a state likely to adversely affect the measurement value such as abnormal attachment of the device is detected, or the like).

In a case where there is a measurement error, the error is immediately notified, and thus it is possible to increase a possibility that the patient can perform re-measurement within a predetermined time (that is, within a time determined as one opportunity), and to increase a possibility that an appropriate measurement value of one opportunity can be obtained. Such notification may be performed via the patient-side terminal 300.

In addition, the measuring device 400 may also be configured to perform the notification regarding measurement of at least the heart rate and the pulse rate at a predetermined timing every day. The predetermined timing can be morning (within one hour after getting up), night (before going to bed), or the like, and a plurality of predetermined timings may be provided per day. Here, the patient-side terminal 300 may notify that the measurement should be performed instead of the measuring device 400, but it is desirable to notify, by at least one of the patient-side terminal 300 or the measuring device 400, that the measurement of the heart rate and the pulse rate should be performed at a predetermined timing every day. In this manner, it is possible to increase the possibility of acquiring the heart rate and the pulse rate measured at the same measurement opportunity every day.

Various kinds of measurement data such as a heart rate, the heart rate when atrial fibrillation is detected, a pulse rate, presence or absence of arrhythmia, a blood pressure value, and a weight, which are measured by using the measuring device 400, are transmitted to the patient-side terminal 300 by wired or wireless communication together with accompanying information such as information relating to the measurement time. In the case of wireless communication, a near-field wireless data communication standard such as Bluetooth (trade name) or infrared communication can be adopted as a communication interface used between the measuring device 400 and the patient-side terminal 300.

The measuring device 400 may not include a communication means. In this case, the patient P may manually input the measurement data (and the measurement date and time information) to the patient-side terminal 300, and the information may be transmitted to the server device 100.

The patient-side terminal 300 may also have the function of the measuring device 400. For example, in a case where the patient-side terminal 300 is a wearable terminal attached to the patient P, and when a measurement function is provided in the wearable terminal, the patient-side terminal 300 can also serve as the measuring device 400. Alternatively, on the contrary, for example, the stationary measuring device 400 may have a function as an information processing terminal and also serve as the patient-side terminal 300.

### Flow of Information Processing in System

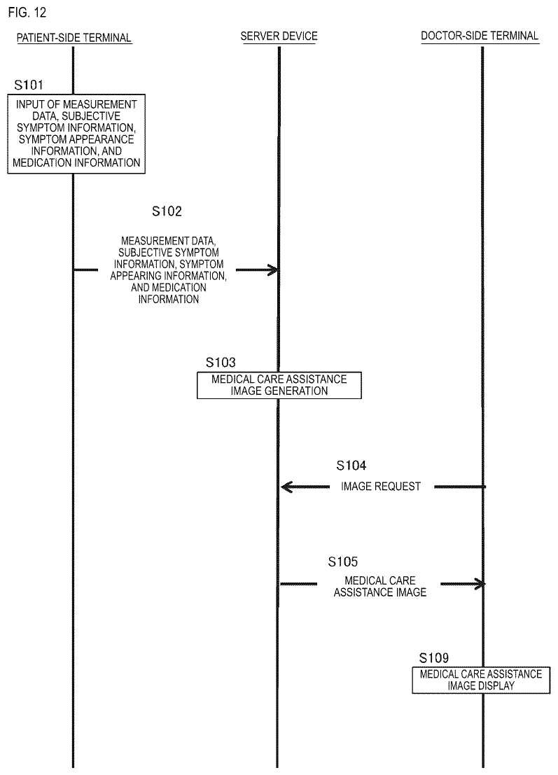

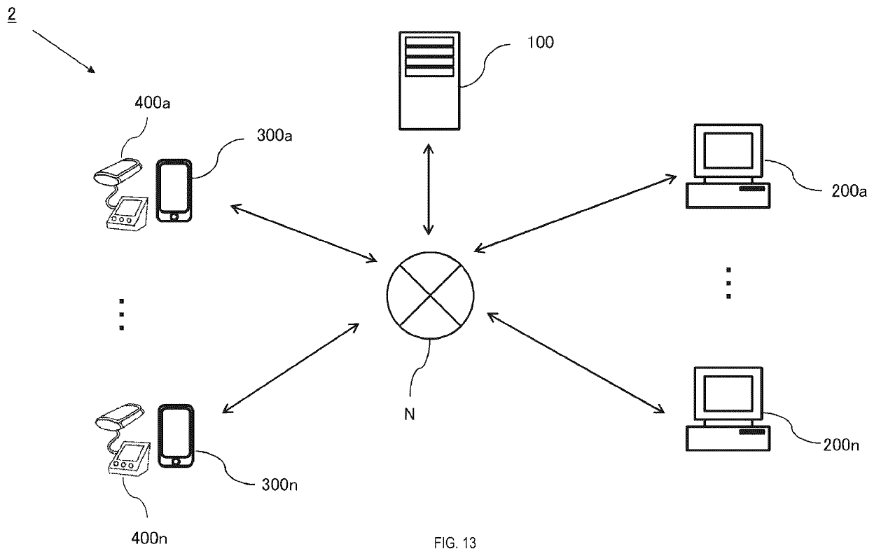

Next, a flow of information processing performed in the medical care assistance system 1 according to the present example having the above-described configuration will be described. FIG. 12 is a diagram illustrating the flow of information exchange and processing performed in the medical care assistance system 1. As illustrated in FIG. 12, first, the measurement data, the symptom appearing exercise information, the subjective symptom information, the medication information, and the like obtained by the measurement of the patient P using the measuring device 400 are input to the patient-side terminal 300 (S101). These pieces of information are transmitted from the patient-side terminal 300 to the server device 100 each time or collectively for a predetermined period (for example, one week) (S102).

In the server device 100, the received various kinds of information are stored in the storage means 130, and a medical care assistance image is generated based on the information (S103).

Thereafter, the physician transmits request information of the medical care assistance image to the server device 100 via the physician-side terminal 200 (S104). Upon receiving the request, the server device 100 provides the medical care assistance image to the physician-side terminal 200 (S105), and the medical care assistance image is displayed on the output means 230 of the physician-side terminal 200 (S106). Here, the data of the medical care assistance image may be transmitted to the physician-side terminal 200 and stored in the storage means 240 of the physician-side terminal 200, or the medical care assistance image may be provided in the form of SaaS and the image data may not be stored. The content of the medical care assistance image is as described above.

As described above, according to the medical care assistance system 1 of the present example, the physician can refer to the medical care assistance image in which the information related to the subjective symptom of the heart failure patient and the transition of the estimated severity are indicated on the time axis common to the measurement data of the biological information. According to such a screen, it is possible to efficiently and easily comprehend the transition of the disease condition and the most recent state of the patient, and it is possible to efficiently diagnose the patient by suppressing an inefficient medical interview at the time of each medical examination.

### Modified Examples

In the above-described example, in a case where measurement is performed at a plurality of opportunities in one day and measurement values at the plurality of opportunities are stored in the storage means 130, as a method of determining one opportunity performed by the daily measurement value calculation unit 112 to calculate a daily heart rate and pulse rate, an example of using a measurement value at a measurement opportunity at a timing set in advance has been described. However, the method of determining one opportunity from a plurality of measurement opportunities is not limited thereto. Specifically, one opportunity may be calculated by another method as described below.

### Modified Example 1

For example, the daily measurement value calculation unit 112 may determine, as one opportunity for daily heart rate and pulse rate calculation, a measurement opportunity at which the deviation of the heart rate and the pulse rate measured in one opportunity among the plurality of measurement opportunities is large. According to this, it is possible to clarify the difference between the heart rate and the pulse rate plotted in the heart rate/pulse rate information region HP, and it is possible to easily attract the attention of the physician.

### Modified Example 2

Alternatively, the daily measurement value calculation unit 112 may determine, as one opportunity for daily heart rate and pulse rate calculation, a measurement opportunity at which a time difference between the measurement times of the heart rate and the pulse rate measured in one opportunity among the plurality of measurement opportunities is the smallest. In a case where a healthy person simultaneously (and accurately) measures the heart rate and the pulse rate, it is assumed that these values are equal to each other. Therefore, it is possible to estimate the deterioration of the symptom or the disease condition of the patient from the deviation between the heart rate and the pulse rate in the same measurement opportunity. Therefore, it is desirable that the time difference between the measurements of the heart rate and the pulse rate is smaller (i.e., closer to the same time).

### Modified Example 3

Alternatively, in a case where there are a plurality of measurement opportunities per one day and the heart rate or the pulse rate is measured a plurality of times in each opportunity, the daily measurement value calculation unit 112 may determine, as one opportunity for daily heart rate and pulse rate calculation, a measurement opportunity at which a difference between each heart rate or a difference between each pulse rate acquired at a plurality of times in each opportunity is the smallest. This is because, in such a measurement opportunity, there is a high possibility that the biological information is measured in a more appropriate (less adverse influence) state.

### Modified Example 4

In addition, the daily measurement value calculation unit 112 may determine, as one opportunity for daily heart rate and pulse rate calculation, a measurement opportunity at which a measurement state that is likely to adversely affect the measurement is not detected at the time of measurement of the heart rate and the pulse rate (that is, a measurement opportunity for which there is no additional information indicating such a state in the measurement value).

### Other Points

The description of each example described above is merely illustrative of the present invention, and the present invention is not limited to the specific embodiments described above. Within the scope of the technical idea of the present invention, various modifications and combinations may be made. For example, in the above-described example, a configuration including one of each the physician-side terminal 200 and the patient-side terminal 300 has been described. However, as illustrated in FIG. 13, the present invention can be applied to a medical care assistance system 2 including a plurality of physician-side terminals 200a to 200n and/or a plurality of patient-side terminals 300a to 300n.

The medical care assistance image generation unit 118 may generate a medical care assistance image including a list indicating the contents of the data table illustrated in FIG. 3. If it is possible to refer to the medical care assistance image including such a list at the time of medical examination, the physician can more efficiently perform a medical interview for diagnosing the severity of the patient.

In the above-described example, the NYHA classification is exemplified as the information indicating the severity of heart failure, but the present invention is not necessarily limited thereto. For example, an American heart association/American college of cardiology (ACC/AHA) stage classification or the like may be used as the information indicating the severity. In the above example, the disease of medical care target is heart failure, but the medical care target is not limited thereto. For example, the present invention can also be applied to medical care for hypertensive patients.

### REFERENCE NUMERALS LIST

1, 2 . . . Medical care assistance system
100 . . . Server device
110, 210, 310 . . . Control unit
120, 240, 340 . . . Storage means
130, 250, 350 . . . Communication means
200 . . . Physician-side terminal
220, 320 . . . Input means
230, 330 . . . Output means
300 . . . Patient-side terminal
400 . . . Measuring device
P . . . Patient
N . . . Communication network
OV . . . Summary information region
MM . . . Minimum METs information
W . . . Weight information region
NT . . . Estimated NYHA classification transition region
SB . . . Estimated severity display bar
S . . . Subjective symptom information region
ME . . . Medication information region
BP . . . Blood pressure information region
HP . . . Heart rate/pulse rate information region

1. A medical care assistance system comprising:
a biological information acquisition means configured to acquire biological information of a patient including a heart rate of one or more opportunities and a pulse rate of one or more opportunities of a patient to be managed for each day;
a daily measurement value calculation means configured to calculate one heart rate and one pulse rate of the patient for each day based on the biological information and a predetermined calculation rule;
a medical care assistance image generation means configured to generate a medical care assistance image including a heart rate/pulse rate graph in which the one heart rate and one pulse rate for each day are plotted in one graph area having a time for one axis and a rate for another axis; and
an output means configured to output the medical care assistance image.

2. The medical care assistance system according to claim 1, wherein
the biological information includes the presence or absence of atrial fibrillation, and
the medical care assistance image generation means generates the medical care assistance image including the heart rate/pulse rate graph plotted together with a heart rate when the atrial fibrillation is detected.

3. The medical care assistance system according to claim 2, wherein
the medical care assistance image generation means generates, when the atrial fibrillation is present, the medical care assistance image in which a mark indicating the presence of the atrial fibrillation is also displayed on the heart rate/pulse rate graph.

4. The medical care assistance system according to claim 1, wherein
the biological information acquisition means also acquires the presence or absence of arrhythmia of the patient for each day, and
the medical care assistance image generation means generates, when the arrhythmia is present, the medical care assistance image in which a mark indicating the presence of the arrhythmia is also displayed on the heart rate/pulse rate graph.

5. The medical care assistance system according to claim 1, wherein
the daily measurement value calculation means determines, with respect to the heart rate and the pulse rate in the one opportunity, a measurement value of first measurement as a measurement value of the one opportunity when further measurement is not performed within 10 minutes from the first measurement, or determines an average value of all measurement values performed within 10 minutes as the measurement value of the one opportunity when further measurement is performed within 10 minutes from the first measurement.

6. The medical care assistance system according to claim 1, wherein
when the biological information acquisition means acquires the heart rate and the pulse rate at a plurality of measurement opportunities per one day,
the daily measurement value calculation means selects one opportunity from the plurality of measurement opportunities and calculates the one heart rate and the one pulse rate for each day based on measurement values of the heart rate and the pulse rate in the selected opportunity.

7. The medical care assistance system according to claim 6, wherein
the daily measurement value calculation means selects a measurement opportunity at a predetermined timing as the one opportunity in the one day, and calculates the one heart rate and the one pulse rate based on a measurement value measured at the one opportunity.

8. The medical care assistance system according to claim 6, wherein
the daily measurement value calculation means selects a measurement opportunity in which a deviation between the heart rate and the pulse rate in one measurement opportunity is the largest as the one opportunity in the one day, and calculates the one heart rate and the one pulse rate based on a measurement value measured at the one opportunity.

9. The medical care assistance system according to claim 6, wherein
the daily measurement value calculation means selects a measurement opportunity in which a time difference between measurement times of the heart rate and the pulse rate in one measurement opportunity is the smallest as the one opportunity in the one day, and calculates the one heart rate and the one pulse rate based on a measurement value measured at the one opportunity.

10. The medical care assistance system according to claim 6, wherein
when the biological information acquisition means acquires the heart rate and the pulse rate at a plurality of measurement opportunities per one day and acquires a plurality of the heart rates or the pulse rates in each opportunity,
the daily measurement value calculation means selects a measurement opportunity in which a difference between a plurality of the heart rates or between a plurality of the pulse rates acquired in each opportunity is the smallest as the one opportunity in the one day, and calculates the one heart rate and the one pulse rate based on a measurement value measured at the one opportunity.

11. The medical care assistance system according to claim 1, wherein
the biological information acquisition means also acquires information related to whether or not a measurement state is likely to adversely affect the measurement at the time of measurement of the heart rate and the pulse rate.

12. The medical care assistance system according to claim 11, wherein
when the heart rate and the pulse rate displayed in the heart rate/pulse rate graph are calculated using a measurement value measured in a measurement state that is likely to adversely affect the value, the medical care assistance image generation means generates the medical care assistance image including the heart rate/pulse rate graph allowing to identify that the heart rate and pulse rate are calculated using a measurement value measured in a measurement state that is likely to adversely affect the value.

13. The medical care assistance system according to claim 6, wherein
the biological information acquisition means also acquires information related to whether or not a measurement state is likely to adversely affect the measurement at the time of measurement of the heart rate and the pulse rate,
when the biological information acquisition means acquires the heart rate and the pulse rate at a plurality of measurement opportunities per one day,
the daily measurement value calculation means selects, as the one opportunity in the one day, a measurement opportunity in which there is no information indicating that the measurement state is likely to adversely affect the measurement at the time of measurement of the heart rate and the pulse rate, and calculates the one heart rate and the one pulse rate based on a measurement value measured in the one opportunity.

14. A medical care assistance device comprising: the biological information acquisition means; the daily measurement value calculation means; and the medical care assistance image generation means, the medical care assistance device constituting at least a part of the medical care assistance system according to claim 1.

15. A recording medium for causing a computer to function as the medical care assistance device according to claim 14.
